(19) United States
(12) Patent Application Publication
(10) Pub. No.: US 2022/0344036 A1
(43) Pub. Date: Oct. 27, 2022

(54) Algorithmic Guided Physician Recommendation for Hospital Bedding Status

(71) Applicant: Deepak Pahuja, Frisco, TX (US)

(72) Inventor: Deepak Pahuja, Frisco, TX (US)

(21) Appl. No.: 17/302,106

(22) Filed: Apr. 23, 2021

(51) Int. Cl. G16H 40/20; G16H 50/70; G06N 5/04; G06N 7/00

(52) U.S. Cl. CPC G16H 40/20; G16H 50/70; G06N 5/04; G06N 7/005

(57) ABSTRACT

The present invention determines the status of hospitalized patient as to whether, they should be treated as inpatient or needs to be provided with observation level of care. The invention ascertains the stratification of patients' status through an algorithm guided automated system. The algorithm based automated patient stratification system is based on a statistical scoring pattern that utilizes raw data of hospitalized patients.

### FIELD OF THE INVENTION

The present invention discusses systems and methods for automatically guiding physician as to the status of hospitalization. Moreover, said invention incorporates an algorithm based scoring system to determine the status and care level for hospitalized patient.

### BACKGROUND OF THE INVENTION

Hospital admission decisions are inherently complex clinical judgments formulated by physicians after careful consideration of numerous factors, including the severity of each patient's condition, the likelihood of adverse events, patient medical history, and hospital bylaws and admissions policies. Medicare policy recognizes that the physician responsible for a patient's care at the hospital is also responsible for deciding whether that patient should be admitted as an inpatient. Concerns regarding the admissions status of patients undergoing short hospital stays intensified in recent years as these stays became targets of Medicare recovery audit contractor (RAC) reviews and the use of observation status as an alternative to inpatient admission increased exponentially. Observation care comprised of a set of well-defined clinically appropriate services that includes short-term treatment, assessment and reassessment before a decision can be made as to whether a patient can be discharged or requires further treatment as an inpatient. In the United States, for example, there exist “two-midnight rule” with hospital inpatient admissions considered reasonable and necessary for patients whose stays cross two midnights.

Despite the criteria set for a patient status, the distinction between inpatient versus observation status is not always clear-cut. Patients undergoing short hospital stays may be treated similarly to inpatients but classified as outpatients receiving observation services. Patients may not be aware that they are under observation or that this designation significantly impacts coverage, payment and cost-sharing expenses.

Apart from the stated unclear admission scenario, there exists lack of specific knowledge of patient risk strata that has a negative bearing on the health care administrators to develop wellness programs with population-specific conditions in mind. Also, forecast of future spends levels, and anticipation of resource needs becomes difficult. It is of great significance, then, to improve upon conventional technological approaches to achieve a greater degree of accuracy and dependability, as to the specific medical intervention applicable to a patient taking the established criteria as well as taking care of the gap in patient information.

In the backdrop of the stated ambiguity of the admission status, select medical management patents in place are as follows:

In U.S. Pat. No. 8,515,780B2 titled, “Systems and methods for risk stratification of patient populations” talks of a statistical processing system includes a server operably configured with program instructions implementing a plurality of statistical models to at least one of (a) predict a health outcome based on questionnaire responses, (b) assist a patient's choice of therapeutic modality based on questionnaire responses, and (c) assess a health risk or status based on questionnaire responses. Also provided is a research agency communicating with the server and contracted to provide the statistical models using a visual interface communicated by the server. The server is configured to analyze requests received from users relating to a plurality of said statistical models to reduce redundancy in requests for patient data.

In US20140095184A1 titled, “Identifying group and individual-level risk factors via risk-driven patient stratification” discusses systems and methods for individual risk factor identification include identifying common risk factors for one or more risk targets from population data. Individuals are stratified into clusters based upon the common risk factors. A discriminability of each of the common risk factors is determined, using a processor, for a target cluster using individual data of the target cluster to provide re-ranked common risk factors as individual risk factors for the target cluster, such that the discriminability is a measure of how a risk factor discriminates its cluster from other clusters.

But present systems for managing population health risks do not harness valuable electronic health record data and claims experience for categorizing patient risks and syncing the same with the stipulated criteria for ascertaining admission status. As a consequence, inaccurate and imprecise risk assignment often results, rendering these patented systems less effective as also redundant in assigning the appropriate admission status. To take care of the stratification of health risk and optimally determining the admission status of patients usage of machine learning techniques harnessing electronic data of patients through random sampling becomes pertinent and cost-effective.

The present effort works toward the stated goal by developing an algorithm based recommendation for hospitalized patient that precisely identifies and recommends appropriate hospital status using random sample.

### SUMMARY OF THE INVENTION

An aspect of the present invention is the determination of the status of hospitalized patient as to whether, they should be treated as inpatient or needs to be provided with observation level of care.

In another aspect of the invention, the stratification of patients' status is ascertained vide an algorithm guided automated system.

In still another aspect of the invention, the said algorithm based automated patient stratification system is based on a statistical scoring pattern that utilizes raw data of hospitalized patients.

Another aspect of the invention is that the said algorithm uses raw data of hospitalized patients obtained in a randomized manner from amongst hospital patients. Random samples of 2000 such patients were analyzed in achieving the desired objective.

In further aspect of the invention, the said frequency of association of hospitalized patients is estimated from the most common “symptom keywords” observed amongst hospitalized patients of various age classes.

In another aspect of the invention, the said analysis of randomized data involves analyses for frequency of association of hospitalized patients against assigned parameters reflected through “keywords” to enable estimate the probability of occurrences, against each of these patient age classes.

An aspect of the invention envisages the overwhelming majority of hospitalized inpatients vis-à-vis the total hospitalized patients thereby making the analysis of the remaining hospitalized patients irrelevant.

Still another aspect of the invention is the assigning of scores to the admissible ranges of the various classes (as reflected through specific keywords) on the basis of their frequency of occurrence of the inpatients.

Further aspect of the invention is the application of logistic regression analysis against the recommendations given to the patients of the specific ages. The use of logistic regression is chosen as an apt depiction of the probabilistic distribution closely resembling that of a sigmoid function.

In another aspect of the invention, the stated algorithm allows creation and execution of a multitude of logistic regression models that regresses relevant independent variable as the patients' age with expert's recommendation as the dependent variable.

In an aspect of the invention usage of Zosyn having a higher association with inpatient recommendation is concluded from the frequent usage of the same, as observed from the random sample analysis.

### BRIEF DESCRIPTION OF THE DRAWINGS

FIG. 1 is a flowchart depicting generation of a summary report using report template.

### DETAILED DESCRIPTION OF THE EMBODIMENTS

Various examples of the invention will now be described. The following description provides specific details for a thorough understanding and enabling description of these examples. One skilled in the relevant art will understand, however, that the invention may be practiced without many of these details. Likewise, one skilled in the relevant art will also understand that the invention can include many other obvious features not described in detail herein.

Before explaining at least one embodiment of the inventive concepts disclosed herein in detail, it is to be understood that the inventive concepts are not limited in their application to the details of the steps or methodologies set forth in the following description or illustrated in the drawings. The inventive concepts disclosed herein are capable of other embodiments, or of being practiced or carried out in various ways. Also, it is to be understood that the phraseology and terminology employed herein is for the purpose of description and should not be regarded as limiting the inventive concepts disclosed and claimed herein in any way.

In the following detailed description of embodiments of the inventive concepts, numerous specific details are set forth in order to provide a more thorough understanding of the inventive concepts. However, it will be apparent to one, of ordinary skill in the art that the inventive concepts within the instant disclosure may be practiced without these specific details. In other instances, well-known features have not been described in detail to avoid unnecessarily complicating the instant disclosure.

Finally, as used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

Referring now to the drawings and more particularly to the automatic hospitalized patient recommendation system in accordance with the inventive concepts disclosed herein is illustrated. The said automatic hospitalized patient recommendation system enables stratification of patients' status through a computerized system employing appropriate algorithm.

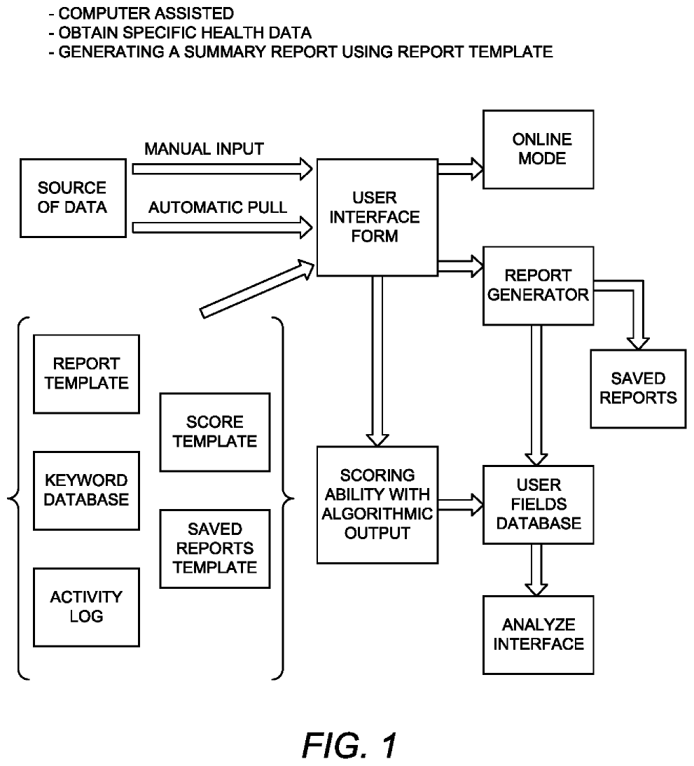

An embodiment of the present invention, is brought forth by FIG. 1 which shows the operational sequence of the algorithm and its functioning. The said sequence of operation involves mining of patients' data utilizing pre-assigned templates, keywords as well as activity logs along with automatic extraction from health database. Utilization of the user interface form of patients enables development of a scoring pattern system and associated extrapolation of expert recommendation to be developed through appropriate analysis described herein.

In another embodiment of the present invention, the language of the said algorithm for automatic processing of hospitalized patients' data had its base from the trend emanating from hospitalized patients admitted to various US hospitals. The algorithm enables collecting a random sample of 2000 from amongst raw hospitalized patients' data for analysis on the basis of “symptom keywords”. The probability of occurrences of each of these “symptom keywords” on the basis of frequency of different classes are thereafter calculated.

Further, the algorithm incorporates appropriate “keyword symptoms” on the basis of the overwhelming presence of the same amongst hospitalized in-patients. The said algorithm allows in-patients to be analyzed intricately rather than the entire gamut of hospitalized patients.

Another embodiment of the invention discusses appropriate algorithm for embodying a scoring pattern based on the frequency of occurrences of the different patient classes. The scoring determines the weight to a specific “keyword symptoms” prevalent amongst the hospitalized in-patients.

Further embodiment talks of regression of the recommendations reflected through appropriate algorithm. On account of the discreteness of the nature of the recommendation and the sigmoid nature of the probabilistic distribution, a logistic regression analysis is deployed. The model regresses relevant independent variables like the age of the patient to estimate pin-point optimal recommendations.

Also, the algorithm analyses from amongst the various recommendation regressed upon, to reveal the type of generic drugs that have the highest usage amongst hospitalized in-patients.

The method of the present invention involves:

Randomly selecting 2000 hospitalized patients in the US hospital. Since in the randomly selected 2000 patients, an overwhelming percentage of patients are that of inpatients, the out patients have been kept out of analysis.

Classifying the patients in different patient classes on the basis of their age or any other factor that may be found probable.

Allocating pre assigned “keyword symptoms” to the different patient classes

Generating respective frequency against keyword symptoms

dividing the hospitalized patient samples into inpatient and observation level categories

Determining the probability of occurrences of different keyword symptoms

Assigning appropriate score for the symptoms on the basis of frequency

Generating recommendations based on inpatient classes

On the basis of the recommendations of the inpatients taken as independent variable and the corresponding independent variable as the age of the patient, a logistic regression is undertaken and the most probable recommendations that can likely happen is inferred.

The present disclosed subject matter may be a system, a method, and/or a computer program product. The computer program product may include a computer readable storage medium (or media) having computer readable program instructions thereon for causing a processor to carry out aspects of the present disclosed subject matter. The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

1. An automated method of physician recommendation for hospital bedding status, comprising the steps:
randomly selecting hospitalized patients;
classifying the patients on the basis of their age;
allocating pre assigned “keyword symptoms” to the different patient classes;
generating respective frequency against the keyword symptoms;
dividing the hostpitalized patient samples into inpatient and observation level categories;
determining the probability of occurrences of different keyword symptoms;
assigning appropriate score for the symptoms on the basis of frequency; and
generating recommendations based on inpatient classes.

2. The method of claim 1, wherein the method further comprises the step of undertaking a logistic regression by inferring the most probable recommendations based on the recommendations of the inpatients' sample taken as dependent variable and the corresponding independent variable as the age of the patient′

3. A non-transitory computer-readable medium having instructions stored thereon, the instructions executable by a processor of a networked device comprising:
mining of patients' data utilizing filled in preassigned templates, symptoms keywords and activity logs;
automatic extraction of requisite patients' data from health database;
developing a scoring pattern system; and
extrapolation of expert recommendation through appropriate analysis.

4. A non-transitory computer-readable medium of claim 3, wherein, the preassigned template is an user interface form.
